(19) United States
(12) Patent Application Publication
(10) Pub. No.: US 2005/0049186 A1
(43) Pub. Date: Mar. 3, 2005

(54) Peptides mimicking the biological activity of steroid hormones and their uses

(76) Inventors: Fortune Kohen, Tel-Aviv (IL); Venkatesh Natarajan, Boston, MA (US); Ron Kasher, Jerusalem (IL); Matityahu Fridkin, Rehovot (IL); Ephraim Katchalski-Katzir, Rehovot (IL)

Correspondence Address: BROWDY AND NEIMARK, P.L.L.C.;624 NINTH STREET, NW, SUITE 300, WASHINGTON, DC, 20001-5303, US

(21) Appl. No.: 10/240,662

(22) PCT Filed: Apr. 3, 2001
(86) PCT No.: PCT/IL01/00304

(30) Foreign Application Priority Data
Apr. 3, 2000 (IL) 135442

(51) Int. Cl. A61K 38/10; A61K 38/08; C07K 7/08

(52) U.S. Cl. 514/9; 514/13; 514/12; 530/326; 530/327

(57) ABSTRACT

A monoclonal antibody to a steroid hormone is used for the isolation from a combinatorial peptide library of a peptide mimicking the biological activity of said steroid hormone. Peptides having estrogenic-like and progestational-like activity were synthesized and assayed for their ability to compete with the respective steroid hormone for binding to the monoclonal antibody and for binding to said steroid hormone receptor. Peptides having estrogenic-like activity were found to be selective to estrogen receptor alpha or beta and thus may have more clinical applications than the non-selective steroid hormone estradiol.

### FIELD OF THE INVENTION

The present invention relates to peptides mimicking the biologic activity of steroidal hormones, to methods for their identification and isolation and pharmaceutical compositions comprising them.
Abbreviations: BSA—bovine serum albumin; CK—creatine kinase; E2—estradiol; ER—estrogen receptor; mAb—monoclonal antibody; PBS—phosphate-buffered saline; RIA—radioimmunoassay;

### BACKGROUND OF THE INVENTION

Estradiol, a steroid hormone, regulates the growth, differentiation and function of diverse tissues, both within and outside the reproductive system. Because of the multiple target organs (e.g. heart, uterus, brain, breast, immune cells, etc) for estrogens and the occurrence of both beneficial and unwanted effects during treatment, the key to improvement in drug therapy is the development of selective estrogen receptor modulators (SERM's) with better tissue selectivity (Warner et al., 1999). Estrogen mediates its effects via the estrogen receptor (ER) that exists as two subtypes, ER α (Greene and Press, 1986) and ER β (Kuiper et al., 1996; Kuiper and Gustafsson, 1997; Nillson, 1998), which differ in the C-terminal domain and in the N-terminal transactivation domain. The two ER isoforms exhibit distinct tissue distribution patterns and differ in their ligand binding ability and transactivational properties (Kuiper et al., 1997).

In the technique of phage-displayed peptide library, a diverse collection of random peptides displayed on the surface of filamentous phages can be screened for phages that bind to a target molecule, such as an antibody or receptor. This method generates phagotopes that have consensus sequences often matching the sequence found in the peptidic antigen used for generating the antibody used as the selector molecule. The advantages of phage displayed peptide libraries for drug discovery have been reported (Devlin J. J., 1990; Lowman, 1997). Numerous groups used these peptide libraries to identify short peptide mimetics of antigenic epitopes (Cortese et al., 1994; Scott and Smith, 1990), ligands for receptors (Balass et al., 1993; Cabilly et al., 1998a; Cabilly et al., 1998b; Yayon et al., 1993), for proteins (eg streptavidin) (Giebel, 1995) and for biotin binders (Saggio, 1993). Phage peptide libraries and other combinatorial peptide libraries have been extensively utilized in the last years for mapping antigenic epitopes using as probes monoclonal antibodies (mAb) to proteinic antigens.

Although phage displayed peptide libraries have been widely used to study protein-protein interactions (Cesareni et al., 1999; DeWitt, 1999), the use of phage peptide library to understand the interaction of proteins with compounds of non-peptidic nature has been limited. Exceptions to this approach are studies related to peptide that minic carbohydrate antigens (Moe et al., 1999; Qiu et al., 1999). To our knowledge, combinatorial peptide libraries have not been used to isolate peptides mimicking the activity of small molecular weight non-peptidic organic compounds such as steroids.

### SUMMARY OF THE INVENTION

The guiding principle of the present invention is based on the assumption that the binding site of monoclonal antibodies (mAb) recognizing a given steroid represents a molecular template for the steroid molecule. Thus when one finds peptides which recognize such a mAb and compete with the steroid for the mAb template, such peptides might mimic the steroid in its molecular structure as well as in its biological characteristics.

The detection of such peptides is facilitated by the use of suitable synthetic or biological libraries. When a steroid receptor is available, the interaction of the lead peptides with their homologous receptor is investigated. Final test of the lead peptide is naturally carried out by their action in vivo in mice or other suitable animals.

It is thus the purpose of the present invention to provide peptides, hereinafter “the peptides of the invention”, that mimic the biological activity of steroid hormones.

The present invention thus provides a synthetic peptide that mimics the biological activity of a steroid hormone, selected from:

(i) a peptide exhibiting a steroid hormone-like biological activity;

(ii) a peptide obtained from (i) by deletion of one or more amino acid residues;

(iii) a peptide obtained by addition to a peptide (i) or (ii) of one or more natural or non-natural amino acid residues;

(iv) a peptide obtained by replacement of one or more amino acid residues of a peptide (i) to (iii) by the corresponding D-stereomer, by another natural amino acid residue or by a non-natural amino acid residue;

(v) a chemical derivative of a peptide (i) to (iv);

(vi) a cyclic derivative of a peptide (i) to (v);

(vii) a dual peptide consisting of two of the same or different peptides (i) to (vi), wherein the peptides are covalently linked to one another directly or through a spacer; and

(viii) a multimer comprising a number of the same or different peptides (i) to(vi).

In one embodiment, the steroid hormone is an estrogen such as estradiol (17β-estradiol), estrone and estriol and the peptide of the invention has estrogenic-like activity.

In another embodiment, the steroid hormone is a progestogen such as progesterone and the peptide of the invention has progestational-like activity.

In a further embodiment, the steroid hormone is an androgen such as testosterone and the peptide of the invention has androgenic-like activity.

In still a further embodiment, the steroid hormone is a corticoid such as cortisone, hydrocortisone and corticosterone and the peptide of the invention has adrenocorticoid-like activity.

The present invention further provides novel conjugates of the estrogenic peptides of the invention for detection of estrogen receptors on tumor cells, particularly breast cancer cells. The conjugates may be formed with fluorescent markers or with chelating agents such as pentetic acid (DTPA) and either particles of a paramagnetic element such as gadolinium or of a radioactive element such as In⁺⁺⁺. In addition, the invention further provides novel conjugates of the estrogenic peptides of the invention with chemotherapeutic drugs such as adriamycin and daunomycin, for affinity targeting and treatment of estrogen-sensitive tumors, particularly breast cancer.

In another aspect, the present invention relates to the use of a monoclonal antibody to a steroid hormone for the isolation from a combinatorial peptide library, of a peptide exhibiting said steroid hormone biologic-like activity.

The present invention further provides a method for screening a combinatorial peptide library for the identification of a peptide exhibiting a steroid hormone biologic-like activity, which comprises:

(i) providing a monoclonal antibody (mAb) with high affinity and specificity to the steroid hormone investigated;

(ii) screening a combinatorial peptide library with said monoclonal antibody of (i) for the identification of peptides that bind specifically to said mAb;

(iii) isolating said peptides and testing them for competition with said steroid hormone for binding to said mAb in vitro and for binding to said steroid hormone receptor in vitro; and

(iv) identifying the peptides that mimic said steroid hormone activity as being those that successfully compete with said steroid hormone for binding to its respective mAb and receptor.

The invention further provides pharmaceutical compositions comprising a pharmaceutically acceptable carrier and a peptide of the invention.

### BRIEF DESCRIPTION OF THE FIGURES

FIG. 1 shows dose response curves for estradiol (squares) and the estrogen-like peptides 1 (triangles) and 2 (circles) in a competitive binding radioimmunoassay (RIA).

FIG. 2 shows dose response curves for estradiol (squares) and the estrogen-like peptides 1 (triangles) and 2 (circles) using a receptor binding assay for estrogen receptor (ER) alpha.

FIG. 3 shows dose response curves for estradiol (squares) and the estrogen-like peptides 1 (triangles) and 2 (circles) using a receptor binding assay for ER beta.

FIG. 4 shows dose response curves for progesterone (circles) and the progestational-like peptides 3 (triangles) and 4 (squares) in a competitive binding radioimmunoassay (RIA).

### DETAILED DESCRIPTION OF THE INVENTION

According to the invention, the possibility of employing combinatorial peptide libraries was explored to identify peptides that display steroid hormone-like activity. Such an approach offers dual advantage; first, from the structural point of view, it is of interest to test whether a steroid, a hydrophobic polycyclic non-water soluble compound, can be mimicked by a water soluble peptide. Second, if it does, from the application point of view, this allows the preparation of biased combinatorial libraries of chemical and biological origin.

According to the present invention, it is possible to develop peptides with selective activity, for example peptides selective to ER α and/or ER β with appropriate tissue selectivity profiles. The selectivity shown herein for the peptides having estrogenic-like activity is a very important characteristic and advantage of such peptides of the invention, particularly vis-à-vis estradiol itself that is not selective.

In one embodiment of the present invention, we used a specific mAb to estradiol, clone 15, prepared years ago in the laboratory of one of the inventors, to screen a phage-displayed random peptide library to identify a 15-mer peptide that displays estrogenic-like activity in both antibody and receptor binding assays in vitro and in causing an increase in the specific activity of creatine kinase (CK) in rat tissues in vivo. CK was chosen as a response marker since we have previously shown that CK can be used as a response marker for estrogenic activity in vivo in rat animal models and in vitro in skeletal cells (Somjen, 1998; Somjen et al., 1996). Moreover, CK is related to changes in cell division and can be used as a marker for the interaction of estradiol with the functional estrogen receptor (Malnick, 1983).

Thus, this 15-mer peptide exhibiting estrogenic-like activity, initially called H5 and herein in the specification and claims identified as peptide 1, was isolated from a 15-mer phage-peptide library with anti-estradiol mAb clone 15, then sequenced and synthesized by standard methods. An extended version of the peptide 1 was then synthesized and converted to the cyclic peptide 2.

Peptides 1 and 2 have the following sequences:

| | |
|---|---|
| Leu-Pro-Ala-Leu-Asp-Pro-Thr-Lys-Arg-Trp-Phe- | (1) |
| Phe-Glu-Thr-Lys | |
| Cyclic [Cys-Ala-Glu-Leu-Pro-Ala-Leu-Asp-Pro- | (2) |
| Thr-Lys-Arg-Trp-Phe-Phe-Glu-Thr-Lys-Pro-Pro- | |
| Pro-Pro-Cys] | |

Both peptides 1 and 2 were then tested in competitive assays with estradiol for the binding sites of the clone 15 and for binding with the estradiol receptor, thus finding that they bind to the estradiol receptor alpha but not to estradiol receptor beta. In vivo, the peptides increased the specific activity of creatine kinase in some rat tissues while estradiol caused an increase in all rat tissues.

Peptide 1 was then shortened at the N- and/or C-terminal to give peptides with shorter sequence which bind mAb-15 and ER. Then, one or more amino acid residues of the sequences were replaced by different residues thus obtaining the peptides presented in Tables 3 to 5 in Example 8 hereinafter. These peptides were evaluated in terms of binding to anti-estradiol mAb clone 15 and some of them were evaluated also in terms of binding to ER α and β. From the results obtained with these peptides it can be concluded that peptides having estrogenic-like activity derived from peptide 1 should have at least 6 amino acid residues and contain the core peptide WFX₁E wherein X₁ is F or Y.

Thus, in one preferred embodiment, the invention relates to a peptide having estrogenic-like activity of at least 6 amino acid residues of the sequence:
X₄-X₂-Trp-Phe-X₁-Glu-X₃
wherein
X₁ is Phe or Tyr;
X₂ is Lys-Arg-, Ala-Arg-, Lys-Ala-, Val-Arg-, Lys-Pro-, Val-Ser-, or Ile-Arg-;
X₄ is hydrogen or Thr-, Pro-Thr-, Asp-Pro-Thr-, Leu-Asp-Pro-Thr-, Ala-Leu-Asp-Pro-Thr-, Pro-Ala-Leu-Asp-Pro-Thr-, Leu-Pro-Ala-Leu-Asp-Pro-Thr-, or Cys-Ala-Glu-Leu-Pro-Ala-Leu-Asp-Pro-Thr-; and
X₃ is hydroxyl, Thr, -Thr-Lys, or -Thr-Lys-Pro-Pro-Pro-Pro-Cys; and cyclic derivatives thereof.

In preferred embodiments, the anti-estrogenic peptide is selected from the peptides herein designated peptides 1, 2, 44, A-43, A-44, 39, 21, B34 and B37 of the sequences:

| | |
|---|---|
| Leu-Pro-Ala-Leu-Asp-Pro-Thr-Lys-Arg-Trp-Phe- | (1) |
| Phe-Glu-Thr-Lys | |
| Cyclic [Cys-Ala-Glu-Leu-Pro-Ala-Leu-Asp-Pro- | (2) |
| Thr-Lys-Arg-Trp-Phe-Phe-Glu-Thr-Lys-Pro-Pro- | |
| Pro-Pro-Cys] | |
| Lys-Arg-Trp-Phe-Phe-Glu | (44) |
| Ala-Arg-Trp-Phe-Phe-Glu | (A43) |
| Lys-Ala-Trp-Phe-Phe-Glu | (A44) |
| Val-Arg-Trp-Phe-Phe-Glu | (39) |
| Lys-Pro-Trp-Phe-Phe-Glu | (21) |
| Val-Ser-Trp-Phe-Phe-Glu | (B34) |
| Ile-Arg-Trp-Phe-Phe-Glu | (E37) |

It is to be understood that any combinatorial peptide library can be used according to the invention. The examples herein show the use of phage-displayed random peptide library but other libraries such as combinatorial synthetic peptide libraries can be used.

According to the invention, once a peptide that binds the monoclonal antibody is identified, it is then synthesized by standard peptide synthesis methods and tested in vitro in competitive assays with the steroid hormone for the binding sites of the respective monoclonal antibody and for the receptor, and then in vivo for testing the steroid hormone-like activity.

Peptides of the invention having progestational-like activity include, but are not limited to, 15-mer and 16-mer peptides, analogs, derivatives and cyclic forms thereof such as, for example, the peptides herein identified as peptides 3 and 4 of the sequences:
Val-Asn-His-Pro-Trp-Asp-Gln-Ala-Gln-Phe-Leu-Ser-Thr-Ile (3)
Ser-Asn-Pro-Phe-Cys-Gln-Thr-Asp-Gly-Asp-Cys-His-Val-His-Thr (4)

As mentioned above, the term “peptides of the invention” as used herein includes: (i) synthetic peptides having the biological activity of a steroid hormone such as those disclosed herein; (ii) peptides obtained from (i) by deletion of one or more amino acid residues; (iii) peptides obtained by addition to peptides (i) or (ii) of one or more natural or non-natural amino acid residues; (iv) peptides obtained by replacement of one or more amino acid residues of peptides (i) to (iii) by the corresponding D-stereomer, by another natural amino acid residue or by a non-natural amino acid residue; (v) chemical derivatives of the peptides (i) to (iv); (vi) cyclic derivatives of peptides (i) to (v); (vii) dual peptides consisting of two of the same or different peptides (i) to (vi), wherein the peptides are covalently linked to one another directly or through a spacer; and (viii) multimers comprising a number of the same or different peptides (i) to(vi), as long as the peptides (ii) to (viii), also herein referred to sometimes by the terms “peptide derivative” or “peptide analogue”, present substantially the same biological activity of the parent peptide (i).

Typically, modifications are made that retain the steroid hormone-like activity of the parent peptide (i). Any of the above modifications may be utilized alone or in combination, provided that the modified sequence retains the steroid hormone-like activity of the parent peptide, identified by means of the appropriate assays.

Deletion of amino acid residues or addition of one or more natural or non-natural amino acid residues may be made at the N- or the C-terminal of the parent peptide. Substitutions include replacement of the natural amino acid residues by the corresponding D-amino acid residue, for example to increase blood plasma half-life of a therapeutically administered peptide, or by different natural amino acid residues or by non-natural amino acid residues. Thus, the peptide or peptide derivative of the invention may be all-L, all-D or a D,L-peptide.

A “chemical derivative” of a peptide of the invention includes, but is not limited to, a derivative containing additional chemical moieties not normally a part of the peptide provided that the derivative retains the steroid hormone function of the peptide. Examples of such derivatives are: (a) N-acyl derivatives of the amino terminal or of another free amino group, wherein the acyl group may be either an alkanoyl group, e.g. acetyl, hexanoyl, octanoyl, or an aroyl group, e.g. benzoyl; (b) esters of the carboxy terminal or of another free carboxy or hydroxy groups; (c) amides of the carboxy terminal or of another free carboxy groups produced by reaction with ammonia or with a suitable amnine, resulting in the C-terminus or another carboxy group being in the form —C(O)—NH—R, wherein R may be hydrogen, C1-6 alkyl, such as methyl, ethyl, propyl, isopropyl, butyl, isobutyl, pentyl or hexyl, aryl such as phenyl, and aralkyl such as benzyl, such amidation being advantageous in providing additional stability and possibly enhanced activity to the peptide; (d) glycosylated derivatives; (e) phosphorylated derivatives; (f) derivatives conjugated to lipophilic moieties e.g. caproyl, lauryl, stearoyl; and (g) derivatives conjugated to an antibody or other cellular ligands. Also included among the chemical derivatives are those derivatives obtained by modification of the peptide bond —CO—NH—, for example by (a) reduction to —CH₂—NH—; (b) alkylation to —CO—N (alkyl)-; (c) inversion to —NH—CO—.

The term “cyclic peptides” as used herein refers to cyclic derivatives containing either an intramolecular disulfide bond, i.e. —S—S—, an intramolecular amide bond, i.e. —CONH— or —NHCO—, or intramolecular S-alkyl bonds, i.e. —S—(CH₂)ₙ—CONH— or —NH—CO(CH₂)ₙ—S—, wherein n is 1 or 2. The cyclic derivatives containing an intramolecular disulfide bond may be prepared by conventional solid phase synthesis while incorporating suitable S-protected cysteine or homocysteine residues at the positions selected for cyclization such as the amino and carboxy terminals of the peptides, with the option of including spacing residues, such as (Ala)ₙ, (Gly)ₙ where n is from 1 to 4, or non-natural amino acids such as 6-aminocaproic acid, between the terminal residue and the linking residue. The linking residues may then be linked together using known techniques to form cyclicized peptide derivatives. For example, the peptide 2 was prepared by elongation from linear peptide 1 and cyclized according to methods known in the art for formation of a disulphide bond. Following completion of the chain assembly, cyclization can be performed either by selective removal of the S-protecting groups with a consequent on-support oxidation of free corresponding two SH-functions, to form S—S bonds, followed by conventional removal of the product from the support and appropriate purification procedure, or by removal of the peptide from the support along with complete side-chain deprotection, followed by oxidation of the free SH-functions in highly dilute aqueous solution. The cyclic derivatives containing an intramolecular amide bond may be prepared by conventional solid phase synthesis while incorporating suitable amino and carboxyl side-chain protected amino acid derivatives at the positions selected for cyclization. The cyclic derivatives containing intramolecular —S-alkyl bonds may be prepared by conventional solid phase synthesis while incorporating an amino acid residue with a suitable amino-protected side chain, and a suitable S-protected cysteine or homocysteine residue at the positions selected for cyclization. Cyclic peptides may be prepared also as backbone cyclic peptides as described in the literature (Gilon et al., 1991). A backbone cyclization is a method developed to impose conformational constraints on peptides by interconnecting the peptide backbone atoms (N or C^{α}) to each other, to side chains, or to amino and carboxy terminals (Bitan et al., 1997; Gilon et al., 1991)

A “dual peptide” according to the invention consists of two the same or different peptides or peptide derivatives of the invention covalently linked to one another directly or through a spacer such as by a short stretch of alanine residues or by a putative site for proteolysis by cathepsin (see U.S. Pat. No. 5,126,249 and European Patent No. 495,049 with respect to such sites). This will induce site-specific proteolysis of the preferred form into the two desired analogues.

“Multimers” according to the invention consist of polymer molecules formed from a number of the same or different peptides or derivatives thereof. The polymerization is carried out with a suitable polymerization agent, such as 0.1% glutaraldehyde (Audibert et al. (1981) Nature, 289: 593)

The peptides of the invention, in addition to mimicking the steroid hormone activity, may form the basis of novel pharmaceutical agents providing significant advantages over currently available steroid drugs such as estrogenic and anti-estrogenic drugs. Since these peptides, in contrast to steroid hormones, contain reactive amino acids such as a lysine group, novel pharmaceutical agents can be obtained by conjugation to markers, e.g. fluorescent molecules, paramagnetic particles, or radioactive tags, for use in prognosis of treatment of hormone-dependent carcinomas, or to chemotherapeutic drugs such as adriamycin or daunomycin, for reduction of the amount of the toxic antineoplastic agent in cancer treatment.

In endocrine therapy of cancers with hormone receptors on their cells such as breast, endometrium, ovary cancers, hormones are used for palliative therapy of the tumors. For example, tamoxifen, an anti-estrogen oral hormone, can bind to estrogen receptors on breast cancer cells and is used in palliative therapy of breast cancer, being particularly effective for metastatic breast cancer in the postmenopausal woman. Therefore, the detection of the presence of estrogen receptors in said tumor cells is a useful tool for the prognosis of the endocrine therapy.

Thus, in one embodiment, the invention further provides novel conjugates of the peptides of the invention having estrogenic-like activity, such as peptides 1 and 2, for detection of estrogen receptors on tumor cells, particularly breast cancer cells. The conjugates may be formed with fluorescent markers such as fluorescein isothiocyanate (FITC), in which case the estrogen receptors are detected in vitro by immunofluorescence of breast cancer cells or tissue, or with chelating agents such as pentetic acid (DTPA) linked to either particles of a paramagnetic element such as gadolinium or of a radioactive element such as In⁺⁺⁺, for in vivo magnetic resonance imaging (MRI) or radioimmmunodetection, respectively, of breast cancer cells or tissue.

Thus the invention further provides an in vitro method for detection of estrogen receptors in a cancer patient, particularly breast cancer, for prognosis of endocrine treatment of said patient, which comprises reacting a suitable sample, such as a paraffin section, of tissue, e.g. breast tissue, obtained from said patient, with an estrogenic-like peptide of the invention conjugated to a fluorescent marker such as fluorescein isothiocyanate (FITC), and detecting the estrogen receptors by immumofluorescence of said tissue, the strong presence of fluorescence in the nucleus of the cells of said tissue indicating the presence of estrogen receptors.

In another embodiment, the invention further provides a method for detection of estrogen receptors in a cancer patient, particularly breast cancer, for prognosis of endocrine treatment of said patient, which comprises injecting to said patient a conjugate of an estrogenic-like peptide of the invention with DTPA and gadolinium, and performing MRI of said patient, whereby the localization of gadolinium in the breast tissue indicates the presence of estrogen receptors.

In still another embodiment, the invention further provides a method for detection of estrogen receptors in a cancer patient, particularly breast cancer, for prognosis of endocrine treatment of said patient, which comprises injecting to said patient a conjugate of an estrogenic-like peptide of the invention with DTPA and a radioactive element such as In⁺⁺⁺, and performing radioactive imaging of said patient, whereby the localization of the radioactive element in the breast tissue indicates the presence of estrogen receptors.

In addition, the invention further provides novel conjugates of the estrogenic peptides of the invention with chemotherapeutic drugs such as adriamycin and daunomycin, for affinity targeting and treatment of estrogen-sensitive tumors, particularly breast cancer. The conjugates allow localization of the chemotherapeutic drug and therapy with a lower amount of the toxic drug.

The peptides and peptide derivatives of the invention are obtained by any method of peptide synthesis known to those skilled in the art, such as for example by solid phase peptide synthesis.

The present invention is also directed to pharmaceutical compositions comprising a pharmaceutically acceptable carrier and at least one peptide or peptide derivative of the invention having steroid hormone-like activity. The pharmaceutical composition will be administered according to known modes of peptide administration, including oral, intravenous, subcutaneous, intraarticular, intramuscular, inhalation, intranasal, intrathecal, intradermal, transdermal or other known routes. The dosage administered will be dependent upon the age, sex, health condition and weight of the recipient, and the nature of the effect desired.

The peptides of the invention for use in therapy are typically formulated for administration to patients with a pharmaceutically acceptable carrier or diluent to produce a pharmaceutical composition. The formulation will depend upon the nature of the peptide and the route of administration but typically they can be formulated for topical, parenteral, intramuscular, intravenous, intraperitoneal, intranasal inhalation, lung inhalation, intradermal or intra-articular administration. The peptide may be used in an injectable form. It may therefore be mixed with any vehicle which is pharmaceutically acceptable for an injectable formulation, preferably for a direct injection at the site to be treated, although it maybe administered systemically.

The pharmaceutically acceptable carrier or diluent may be, for example, sterile isotonic saline solutions, or other isotonic solutions such as phosphate-buffered saline.

The peptides of the present invention may be admixed with any suitable binder(s), lubricant(s), suspending agent(s), coating agent(s), solubilising agent(s). It is also preferred to formulate the peptide in an orally active form.

Tablets or capsules of the peptides may be administered singly or two or more at a time, as appropriate. It is also possible to administer the peptides in sustained release formulations.

Typically, the physician will determine the actual dosage which will be most suitable for an individual patient and it will vary with the age, weight and response of the particular patient. There can, of course, be individual instances where higher or lower dosage ranges are merited, and such are within the scope of this invention.

Alternatively, the peptides of the invention, can be administered by inhalation or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, at a concentration of between 1 and 10% by weight, into an ointment consisting of a white wax or white soft paraffin base together with such stabilisers and preservatives as may be required.

For some applications, preferably the compositions are administered orally in the form of tablets containing excipients such as starch or lactose, or in capsules or ovules either alone or in admixture with excipients, or in the form of elixirs, solutions or suspensions containing flavouring or colouring agents. For such oral administration, the peptide may preferably formed into microcapsules or nanoparticles together with biocompatible polymers such as polylactic acid and the like.

The compositions (as well as the peptides alone) can also be injected parenterally, for example intracavernosally, intravenously, intramuscularly or subcutaneously. In this case, the compositions will comprise a suitable carrier or diluent. For parenteral administration, the compositions are best used in the form of a sterile aqueous solution which may contain other substances, for example enough salts or monosaccharides to make the solution isotonic with blood.

For buccal or sublingual administration the compositions may be administered in the form of tablets or lozenges which can be formulated in a conventional manner.

The peptides and peptide derivatives of the invention are for use in the treatment of conditions in which steroid hormones are commonly used.

For example, the peptides having estrogenic-like activity may be used for treatment of hormone-dependent cancers such as breast, prostate and colon cancer, in hormone-replacement therapy, in prevention and/or treatment of osteoporosis, etc. In addition, these peptides may be useful for the prevention and/or treatment of degenerative diseases of the central nervous system like Alzheimer's disease and Parkinson's disease as well as those resulting from trauma and stroke in the brain, based on recent findings that estrogens have neuroprotective effects in the adult brain and exert beneficial effects in the treatment of Alzheimer's disease and other neurodegenerative diseases of the central nervous system like Parkinson's disease as well as those resulting from trauma and stroke in the brain (Wang et al., 2001; Munoz and Feldman, 2000; Wise, 2000; Sapolsky and Finch, 2000; Green and Simpkins, 2000). Some of these peptides, unlike estradiol, recognize only ER α or ER β or both receptors. Being more selective than estradiol, these peptides may have more clinical applications. For instance, estradiol is widely used in hormone replacement therapy to help the maintenance of the CNS in postmenopausal women. However, estrogen has unwanted side effects such as an increased risk of uterine cancer. Since the mature uterus has very little ER β, peptides that are selective ER β agonists might be efficient in protecting the brain from age-related neurodegeneration without affecting the uterus.

As progestins, the peptides having progestational-like activity may be used for contraception or in endocrine therapy of breast cancer, uterine fibroids or polycystic ovary syndrome. As corticoid substitutes, the peptides having adrenocorticoid-like activity may be used as anti-inflammatory in all disorders treated by corticoids.

The present invention further relates to a method of treatment of a patient suffering from a disorder that can be treated with a steroid hormone which comprises administering to said patient an effective amount of a peptide or peptide derivative of the invention. In specific embodiments, the method may be used for prevention and or treatment of all conditions mentioned above.

The invention will now be illustrated by the following non-limiting Examples.

### EXAMPLES

Materials and Methods

(i) Monoclonal Antibodies (mAbs): The preparation of specific mAbs to steroids has been previously described (Kohen, 1986; Somjen, 1998). Anti-estradiol mAb clone 15 was derived from a BALB/c female mouse immunized with estradiol-6-(O-carboxymethyl)oxime-bovine serum albumin (E2-6.BSA). Clone 15 belonged to the IgG1 class. Purified IgG fraction was prepared by affinity chromatography on Sepharose-Protein A as previously described (Strassburger and Kohen, 1990). The mAb to progesterone, clone 1E11, was obtained from a CD2 mouse immunized with progesterone-11-alpha hemisuccinate-BSA as previously described (De Boever et al., 1989).
(ii) Biotinylation of anti-estradiol clone 15: Anti-estradiol IgG, clone 15 (100 μg) in NaHCO₃ (100 μl) was reacted with 5 μl of N-hydroxysuccinimide ester of biotin solution in dimethylfomiamide (1 mg/ml) for 2 hours at room temperature. The reaction mixture was then dialysed at 4° C. against phosphate-buffered saline (PBS, pH 7.4). Biotinylation was ensured by binding of the biotinylated mAb on streptavidin-coated plates.
(iii) Screening for monoclonal anti-estradiol (anti-E2) binding phages: The phage-peptide library (a 15-mer library obtained from J. J. Devlin, Cetus Corporation, Emeryville, Calif., USA), was subjected to three rounds of affinity selection against the biotinylated mAb to estradiol, clone 15, as described before (Devlin J. J., 1990), with minor modifications. Petri plates (60 mm, Nunc) were coated with 800 μl streptavidin (10 μg/ml in 0.1M NaHCO₃, pH 9.6) for overnight at 4° C. and then blocked with PBS containing 3% BSA and 0.1 mg/ml streptavidin 1 hour at 37° C. An aliquot of the 15-mer library containing 1×10¹¹ phage particles was incubated with 2.5 μg biotinylated clone 15 in 40 μl PBS containing 0.5% BSA for overnight at 4° C. The streptavidin-coated plates were washed 6 times with PBS containing 0.05% Tween-20 (PBST). The preincubated phage-mAb mixture was diluted to 600 μl with PBST and then transferred to the streptavidin-coated plate. Following incubation for 30 minutes at room temperature with gentle shaking, the plates were washed 10 times with PBST-0.05, over a period of 1 h. Bound phages were eluted with 600 μl 0.1 M glycine.HCl (pH 2.2) containing 1 mg/ml BSA for 10 min and immediately transferred to 50 μl 2 M Tris. An aliquot of the eluate was retained for titration and the rest of the eluate was amplified by infecting log phase culture of E.coli K91 Kan and plated on 150 mm plates LB containing 100 μg/ml ampicillin. After incubation overnight at 37° C., the cells were harvested in 10 ml PBS and centrifuged. The amplified phages in the supernatant were purified by precipitating twice with polyethylene glycol (PEG)/NaCl (Smith and Scott, 1993). The final phage pellet obtained was resuspended in 500 μl PBS, quantified spectrophotometrically and used for subsequent rounds of panning. In order to select high affinity clones the amount of biotinylated mAb was progressively reduced to 1 μg and 0.1 μg in the second and third rounds of panning, respectively. Washing steps were performed with PBS containing 0.5% Tween 20 in the second and third panning.

For titration, 10 μl of an appropriate dilution of the phage was incubated with 100 μl of log phase culture of K91 Kan cells for 10 min at 37° C. and spread on LB plates containing 100 μg/ml of ampicillin.
(iv) Selection of anti-E2 mAb binding phages by biopanning: Well isolated ampicillin-resistant colonies from the third round of panning were individually inoculated into 150 μl of superbroth containing 100 μg/ml of ampicillin in 96-well tissue culture plates (Costar). After incubation overnight at 37° C., the cells were pelleted and the phage-supernatants transferred to another 96-well tissue culture plate and stored at 4° C.

For phage-ELISA, microtiter plates (Maxisorb, Nunc, Neptune, N.J.) were coated overnight at 4° C. with 50 μl rabbit anti-M13 antiserum (1:2000 dilution in 0.1M NaHCO3). Plates were then washed thrice with PBS-0.05T and incubated with 3% blocking buffer (1.5% BSA and 1.5% hemoglobin in PBS) for 1 h at 37° C. Following three washes with PBS-0.05T, 50 μl of phage supernatants (obtained as described above) from each clone were then added to individual wells in the ELISA plate. The plate was incubated for 1 h at 37° C. to capture the phages. The plate was washed three times with PBS-0.05T and incubated with biotinylated anti-estradiol mAb (diluted to 1 μg/ml in 1% blocking buffer) for overnight at 4° C.
(v) Sequencing of phage DNA: Phages from selected clones were purified by PEG/NaCl precipitation, single-stranded DNA was prepared and sequences were determined by a dideoxy chain termination method using an Applied Biosystems (Perkin Elmer) sequenator.
(vi) Synthetic peptides: Peptides were synthesized by the Chemical Services Unit or at the Department of Organic Chemistry of the Weizmann Institute of Science, Rehovot, or at the Hebrew University, Jerusalem, both in Israel, and were purified by known methods.
(vii) Screening for monoclonal anti-progesterone (anti-P) binding phages. Biopanning for anti-P binding phages was performed essentially as described for the screening of anti-E2 binding phages (in (iv) above) with the following modifications: A solution of purified anti-P mAb IgG, clone 1E11 (100 μg/ml in 0.1 M NaHCO_{3,} 100 μl) was added to two wells of a 96 well-microtiter plate and incubated for overnight at 4° C. The unbound solution was discarded and the plates were blocked with PBS containing 3% BSA 1 h at 37° C. Meanwhile, an aliquot of the original peptide library (15-mer linear library from Devlin or 16-mer constrained library from Smith, 10¹¹ phage particles) was incubated with the blocking solution in a total volume of 225 μl for 2 h at 37° C. The wells coated with anti-P mAb were washed 6 times with PBS-0.1T and 100 μl. of preincubated phage library was added to each well. After 4 hours incubation at room temperature, the contents were discarded. The wells were filled with PBS-0.1T, shaken for a minute and the contents were discarded. In the same way, washing was repeated 10 times, following which the bound phages were eluted with 0.1 M glycine.HCl (pH 2.2) containing 1 mg/ml BSA (100 μl /well) for 10 min and immediately transferred to tubes containing 9 μl 2 M Tris. Eluates obtained using each library were individually pooled. Amplification and purification of the phages were done as explained for anti-E2 binding phages. In order to select high affinity clones the coating concentration of mAb was reduced to 1 μg and 0.1 μg per well in the second and third rounds of panning, respectively. The rest of the steps were performed as described for anti-E2 mAb.

### Example 1

Selection of Phages and Peparation of Synthetic Peptides

As described above in Materials and Methods, biotinylated anti-E2 clone 15 was incubated with a 15-mer phage epitope library, and the phage-mAb complexes were captured on streptavidin-coated plates. Bound phages were eluted with 0.1M HCl.glycine pH 2.2, amplified and used for subsequent rounds of panning. After three rounds of panning, phage clones were screened for mAb binding by phage-ELISA. Thirty positive clones were selected and the DNA encoding the displayed peptide were sequenced.

The insert sequences represented three different peptide sequences as shown in Table 1.

TABLE 1
Peptides selected from a 15-mer phage display peptide library
using anti-estradiol mAb, clone 15, as probe.

| | Sequence^{a} | Frequency^{b} |
|---|---|---|
| H5 | LPALDPTKRWFFETK | 21 |
| G5 | AHWNSENTVVGLPSK | 6 |
| H8 | GMQMHQRHVYLSKRP | 3 |

^{a}Amino acid sequences of peptide inserts were deduced from DNA sequencing of the insert of the positive phage clones.
^{b}Frequency denotes the number of phage clones with identical inserts.

There was no consensus among the three selected peptides. Initial binding studies indicated that only peptide H5, herein in the specification and claims identified as peptide 1, recognized anti-E2 mAb clone 15 (see below). Accordingly, we concentrated our studies on this linear peptide 1.

The molecular weight of the linear peptide 1 was confirmed by electron mass spectrometry as being of 1848 (M-1).

### Example 2

Synthesis of the Extended Cyclic Peptide 2

Based on our earlier studies, we found that the affinity of the peptides selected from phage display library can be significantly improved by incorporating flanking aminoacids of the phage coat protein and subsequent cyclization (Venlcatesh et al., 2000), at least for few epitopes which are conformationally constrained. In order to check whether the same is true with the linear peptide 1, we synthesized a derivative, herein designated peptide 2, by flanking peptide 1 with residues -AEC at the N-terminus and -PPPPC at the C-terminus, and air oxidized the peptide to prepare a cyclized peptide 2 having the sequence:

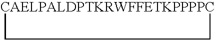

For the synthesis of peptide 2, the extended linear peptide CAELPALDPTKRWFFETKPPPPC (10 mg) was air-oxidized by stirring a 0.25 mg/ml solution of the peptide in water, pH 10.00 (adjusted with ammonium hydroxide) for 48 h at room temperature. Complete oxidation was ensured by estimating the lack of free-SH using Ellman's reagent. Formation of a single monomeric cyclic peptide was ensured by HPLC and by molecular weight determination using electron mass spectrometry. The cyclized peptide 2 showed a molecular weight of 2643 (M−1).

### Example 3

Preparation of Fluorescent Labeled Linear Peptide 1

The linear peptide 1 of the sequence: LPALDPTKRWFFETK contains 2 lysine (K) and 1 glutamic acid (E) residues and can be easily conjugated to several molecules of interest.

For the conjugation with fluorescein, the linear peptide 1 (1.8 mg) was dissolved in 200 μl of 50 mM carbonate-bicarbonate buffer (pH 9.6). Celite-FITC powder (6.4 mg) was added and the reaction mixture was stirred overnight at room temperature and subsequently centrifuged. The filtrate was purified by gel filtration on Sephadex G-10 column using 50 mM Tris-HCl, pH 7.45 as eluant. UV measurement of the eluate at 495 nm indicated that 2 moles FITC were incorporated per mole of the peptide. The labeled peptide was stored at −20° C. in the dark until use. This fluorescein labeled peptide 1 displayed the same binding activity to anti-E2 clone 15 as the original linear peptide 1 (not shown).

### Example 4

Characterization of the Peptides in Terms of Antibody Recognition

ELISA methodology was used first for the initial screening of the various peptides for antibody binding specificity. In this system, the analyte (e.g. estradiol, testosterone, progesterone, estriol) or the peptides and a defined amount of the homologous solid-phase hapten-protein conjugate [e.g. estradiol-6-(O-carboxymethyl) oxime ovalbumin, testosterone-3-(O-carboxymethyl)oxime ovalbumin, progesterone-11a-hemisuccinate ovalbumin or estriol-6-(O-carboxymethyl)oxime ovalbumin] compete for a limited concentration of the specific homologous antibody. The linear peptide 1 and the cyclized peptide 2 at a concentration of 0.5 mM inhibited by 36% the binding of immobilized estradiol ovalbumin conjugate to anti-E2, clone 15, the antibody used in the screening of the phage display peptide library, but not of other high affinity anti-E2 antibodies, clones 8D9, 11B6 and 2F9 (Somjen et al., 1998). On the other hand, E2 used as a control at a concentration of 150 nM inhibited by 30% the binding of estradiol ovalbumin conjugate of all the anti-E2 antibodies that were tested. Moreover, the peptides were not capable of competing with the various solid phase hapten protein conjugates for the binding sites of the homologous anti-steroidal antibodies (eg anti-progesterone, clone 1E11, anti-estriol, clone 9B4, anti-testosterone, clone 5F2). These results indicate that the linear peptide 1 isolated from the phage peptide display library recognizes an epitope of only anti-E2 clone 15 and not of other high affinity anti-steroidal antibodies (data not shown).

### Example 5

The Synthetic Peptides 1 and 2 Compete With Estrogen for the Binding Sites of Anti-E2 Clone 15 in a Competititve Immunoassay System Using Radioactivity or Fluorescence as an End Point.

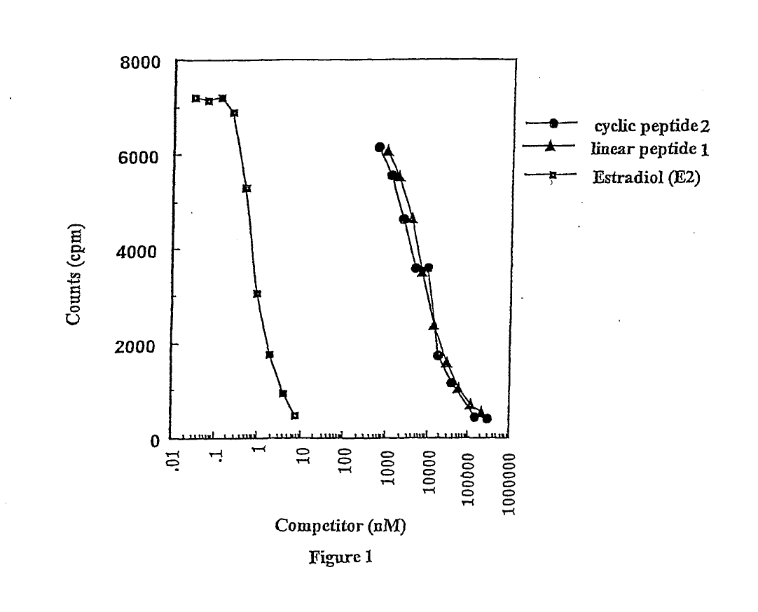

In order to confirm the specific binding activity of the peptides 1 and 2 for anti-E2 mAb, clone 15, a radioimmunoassay system or a time-resolved fluoroimmunoassay system were used where the peptides competed with [³H]-estradiol or with estradiol ovalbumun europium conjugate for the binding sites of clone 15. Dose response curves for estradiol and the peptides using radioimmunoassay are shown in FIG. 1. The two synthetic peptides 1 and 2 competed with [³]H-estradiol for the binding sites of the anti-estradiol mAb, clone 15, with an IC₅₀ of about 5 μM whereas the IC₅₀ for estradiol was <0.8 nM. In contrast to our earlier observation (Venkatesh et al., 2000), we did not observe any improvement in the binding affinity of the cyclic peptide 2 towards anti-E2 15.

### Example 6

Synthetic Peptides 1 and 2 Bind Estradiol Receptor (ER) α but not ER β

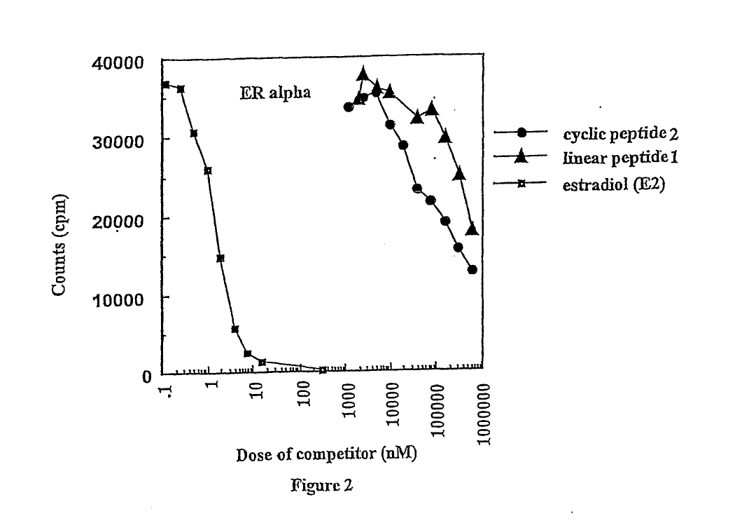

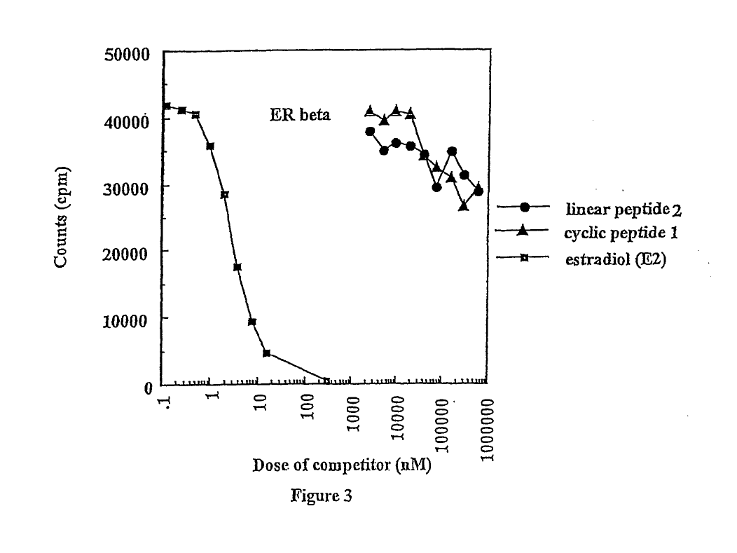

The dose response curves of estradiol and of the linear and cyclic peptides 1 and 2 for binding to ER α and β are shown in FIGS. 2 and 3, respectively. The binding affinity of estradiol for the two receptors was high (IC₅₀ 1 nM). On the other hand, unlike estrogen, the two peptides showed selectivity for binding to ER α, but none to ER β. Interestingly, the cyclic peptide 2 had a relatively better affinity to ER α (IC₅₀ 100 μM) as compared to the linear peptide 1 (IC₅₀ 500 μM), suggesting that the binding of the ligand to the ER is conformation dependent.

### Example 7

The Synthetic Peptides 1 and 2 Induce Creatine Kinase Activity in Rat Tissues in Vivo

Treatment of immature female rats for 4 hours with estradiol (5 μg/rat) or the cyclic peptide 2 (0.5 mg/rat) caused an increase in the specific activity of creatine kinase (CK) in all the tissues (uterus, aorta, and left ventricle) that were examined. On the other hand the linear peptide 1 at this concentration (0.5 mg/rat) caused an increase in CK activity only in the uterus and left ventricle. At higher concentration (2.5 mg/rat) the linear peptide 1 stimulated the CK activity in the epiphysis and aorta as well (data not shown). When rats were treated with a combination of the estrogen receptor antagonist raloxifene with estradiol or with the peptides 1 and 2, the increase in CK activity could be blocked in all the tissues with the exception of the epiphysis where raloxifene could not inhibit the stimulatory activity of the cyclic peptide 2.

Immature female rats were injected with estradiol (5 μg/rat), the cyclic peptide 2 (0.5 mg/rat), the linear peptide 1 (0.5 mg/rat), raloxifene (0.5 mg/rat), the cyclic peptide 2 (0.5 mg/rat) plus raloxifene (0.5 mg/rat), the linear peptide 1 (0.5 mg/rat) plus raloxifene (0.5 mg/rat) or estradiol (5 μg/rat) plus raloxifene (0.5 mg/rat). The control goups received 0.5 ml saline containing 0.5% ethanol or tris-saline alone. The various organs were assayed for CK activity four hours after treatment. The results are shown in Table 2 and are expressed as means ±S.E.M., for n=15, and further expressed as experimental (E) over control (C) where the control is given a value of 1.0.

TABLE 2
Stimulation of the specific activity of creatine kinase by estrogen and synthetic
peptides 1 and 2 in rat tissues in vivo

| | Creatine kinase specific activity | | | | |
|---|---|---|---|---|---|
| | (Experimental/control) Organ | | | | |
| Treatment | Uterus | Diapysis | Epipysis | Aorta | LV# |
| Control | 1 ± 0.13 | 1 ± 0.19 | 1 ± 0.18 | 1 ± 0.13 | 1 ± 0.2 |
| Estradiol | 1.37 ± 0.07** | 1.87 ± 0.1* | 1.37 ± 0.07** | 2.0 ± 0.18* | 1.83 ± 0.12* |
| Peptide 1 | 1.6 ± 0.27* | 1.09 ± 0.17 | 1.10 ± 0.09 | 1.35 ± 0.23 | 1.41 ± 03** |
| Peptide 2 | 1.36 ± 0.11** | 1.39 ± 0.10** | 1.37 ± 0.08** | 1.79 ± 0.12* | 1.61 ± 0.15** |
| Raloxifene | 0.99 ± 0.17 | 1.75 ± 0.13* | 1.41 ± 0.05* | 1.63 ± 0.04* | 1.34 ± 0.11** |
| Raloxifene + peptide 1 | 0.92 ± 0.05 | 1.21 ± 0.18 | 1.04 ± 0.14 | 1.18 ± 0.18 | 1.12 ± 0.19 |
| Raloxifene + peptide 2 | 1.24 ± 0.12 | 1.37 ± 0.31 | 1.56 ± 0.07* | 1.36 ± 0.24 | 0.91 ± 0.20 |
| Raloxifene + estradiol | 1.20 ± 0.11 | 0.78 ± 0.20 | 1.14 ± 0.16 | 1.41 ± 0.36 | 0.75 ± 0.21 |

(Walter et al., 1985)
*p < 0.01;
**p < 0.05; treated vs control
#LV = left ventricle

### Example 8

Peptides Obtained from the Linear Peptide 1 by Deletion of One or More Amino Acid Residues and Their Properties

The linear peptide 1 is 15-amino acid long. We further explored the possibility whether smaller peptides derived from peptide 1 may have antibody binding activity and receptor binding activity. In order to find out the minimum amino acid sequence required in the linear peptide 1 for antibody and receptor binding, several peptides with varying length of from 4 to 14 amino acids long were synthesized, and were first evaluated in terms of binding to anti-estradiol (E2) mAb clone 15. The peptides showing similar or better antibody binding activity than the linear peptide 1 were further evaluated in terms of binding to ERα and β. Table 3 shows the results.

TABLE 3
Peptides derived from peptide 1

| | | Inhibition | Inhibition | |
|---|---|---|---|---|
| | | of binding | of binding | Inhibition of |
| | | of labeled | of labeled | binding of |
| | | E2* to | E2** to | labeled E2** |
| Peptide | Sequence | Anti-E2 | ERα | to ERβ |
| 1 | LPALDPTKRWFFETK | + | + | − |
| 7 | LPALDPTKRWFFET | + | N.E. | N.E. |
| 9 | LPALDPTKRWFEE | + | N.E. | N.E. |
| 15 | LPALDPTKRWFF | − | N.E. | N.E. |
| 16 | LPALDPTKRWF | − | N.E. | N.E. |
| 6 | PALDPTKRWFFETK | + | N.E. | N.E. |
| 8 | ALDPTKRWFFETK | + | N.E. | N.E. |
| 14 | LDPTKRWFFETK | + | N.E. | N.E. |
| 17 | DPTKRWFFETK | + | N.E. | N.E. |
| 18 | PTKRWFFETK | + | N.E. | N.E. |
| 35 | TKRWFFETK | + | N.E. | N.E. |
| 42 | PTKRWFFE | + | N.E. | N.E. |
| 43 | TKRWFFE | + | N.E. | N.E. |
| 44 | KRWFFE | + | + | + |
| 45 | RWFFE | + | − | N.E. |
| 46 | WFFE | + | − | N.E. |

*labeled E2 = [³H]-Estradiol; **labeled E2 = estradiol ovalbumin europium conjugate;
N.E = not evaluated

The results shown in Table 3 indicate that a tetrapeptide with sequence of WFFE is the minimal length required for inhibition of binding of estradiol ovalbumin europium conjugate to anti-estradiol mAb clone 15. However, the tetrapeptide WFFE did not show any inhibition of binding of [³H]-estradiol to the estrogen receptors. On the other hand, the hexapeptide designated 44, of the sequence: KRWFFE, unlike the linear peptide 1, recognized estrogen receptor α as well as β.

Since peptide 44 showed binding activity to the estrogen receptors, we proceeded to evaluate the amino acid residues that are necessary for binding to the estrogen receptors by alanine screen. Six peptides in which every amino acid residue in peptide 44 was replaced by alanine were synthesized and evaluated for antibody binding activity and, those that were positive, also for receptor binding activity. Table 4 shows the results.

TABLE 4
Properties of peptides obtained from peptide
44 (KRWFFE) by alanine screen

| | | Inhibition of | Inhibition of | Inhibition of |
|---|---|---|---|---|
| | | binding of | binding of | binding of |
| | | labeled E2 to | labeled E2 to | labeled E2 to |
| Peptide | Sequence | anti-E2 | ERα | ERβ |
| A-43 | ARWFFE | + | + | − |
| A-44 | KAWFFE | + | + | + |
| A-45 | KRAFFE | − | | |
| A-46 | KRWAFE | − | | |
| A-47 | KRWFAE | − | | |
| A-48 | KRWFFA | − | | |

The results shown in Table 4 indicate that only two peptides (A-43 and A-44) showed inhibition of binding of estradiol ovalbumin europium conjugate to anti-estradiol mAb clone 15. When the estrogen binding ability of these two peptides was evaluated, peptide A-44 recognized both estrogen receptor α and β whereas peptide A-43 recognized only the estrogen receptor α and not β. These results indicate that the lysine residue K at position 1 of the 6-mer peptide is essential for binding to estrogen receptor α and not to estrogen receptor β and the arginine residue R at position 2 of the 6-mer peptide is not essential for binding to estrogen receptor α or β. Moreover, amino acid residues WFEE are essential for binding to anti-estradiol mAb clone 15.

Since peptide 44 showed estrogen receptor binding activity and better inhibition of estradiol ovalbumin europium to anti-estradiol mAb clone 15 than the linear peptide 1, several peptides were synthesized in which each amino acid residue in peptide 44 was replaced by another amino acid residue belonging to the same group of amino acids (e.g. charged, hydrophobic, aromatic, polar, positive, aliphatic, small and tiny). Table 5 shows the results obtained with the peptides showing estrogen like activity.

TABLE 5

| | | IC₅₀ to | IC₅₀ to | |
|---|---|---|---|---|
| Peptide | Sequence | Anti-E2 15 | ER α | IC₅₀ to ERβ |
| Linear 1 | LPALDPTKRWFFETK | 1000 nM | 500 μM | none |
| Cyclic 2 | CAELLPALDPTKRWFFETKPPPPC | 1000 nM | 100 μM | none |
| 44 | KRWFFE | 35 nM | 400 μM | 400 μM |
| A43 | ARWFFE | 40 nM | 500 μM | None |
| A44 | KAWFFE | 40 nM | 250 μM | 250 μM |
| 39 | VRWFFE | 15 nM | <100 μM | <100 μM |
| 19 | KSWFFE | 40 nM | None | None |
| 21 | KPWFFE | 45 nM | 500 μM | >500 μM |
| B33 | VPWFFE | 6 nM | None | None |
| B34 | VSWFFE | 6 nM | 250 μM | 100 μM |
| B35 | VRWFYE | 6 nM | None | None |
| B37 | IRWFFE | 20 nM | None | 250 μM |
| B38 | LRWFFE | 40 nM | None | None |

The results shown in Table 5 indicate that peptides 44, A43, A44, 39, 19, 21, B33, B34, B35, B37, B38 inhibit one and half order of magnitude better than the linear or cyclic peptides 1 and 2 the binding of estradiol ovalbumin europium conjugate to anti-E2 mAb clone 15. Unlike the cyclic peptide 2, peptides 44, A44, 39, 21, B34 recognized both estrogen receptors. Interestingly, peptide B37 recognized only the ERβ and peptides 19, B33, B35 and B38 did not show any binding activity to the estrogen receptors.

When the biological activity of peptide 39 was evaluated in vivo, this peptide stimulated the specific activity of creatine kinase in rat tissues (uterus, left ventricle, aorta, diaphysis, and epiphysis) at a dose of 100 μg/rat, a dose 5 times lower than the cyclic peptide 2 (not shown).

### Example 9

The Synthetic Progestational-like Peptides 3 and 4 Compete with Progesterone for the Binding Sites of Anti-progesterone Clone 1E11 in a RIA System

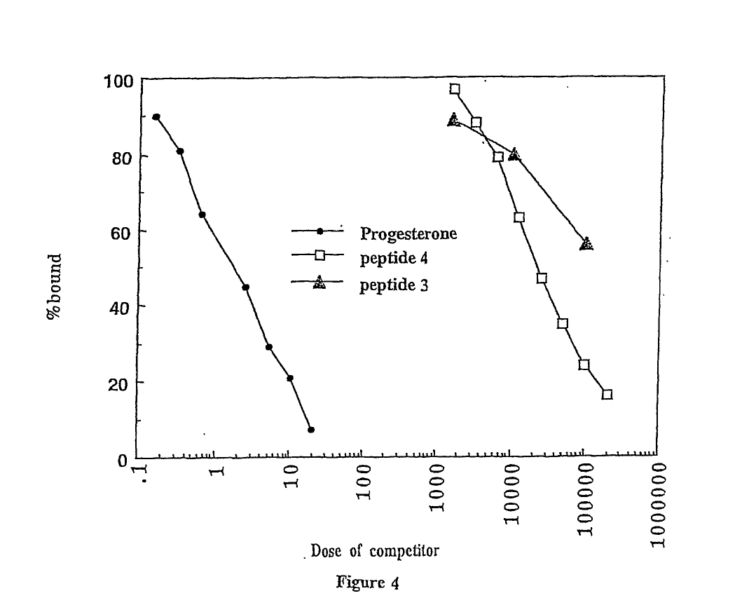

In order to confirm the specific binding activity of the peptides 3 and 4 for the anti-progesterone mnAb clone 1E11, a RIA system where the peptides competed with [³H]-progesterone for the binding sites of clone 1E11 was used. Dose response curves for progesterone and the two synthetic peptides are shown in FIG. 4. The peptide 4 competed with [³H]-progesterone for the binding sites of the anti-progesterone clone 1E11 with IC₅₀ of about 5 μM whereas the IC₅₀ of progesterone itself was <1 nM. On the other hand, the IC₅₀ of peptide 3 was about 100 μM.

References

1. Balass, M., Y. Heldman, S. Cabilly, D. Givol, E. Katchalski-Katzir, and S. Fuchs. 1993. Identification of a hexapeptide that mimics a conformation-dependent binding site of acetylcholine receptor by use of a phage-epitope library. Proc Natl Acad Sci USA. 90:10638-10642.
2. Bitan, G., Muller, D., Kasher, R., Gluhov, E. V. and Gilon, C. 1997. Building units for N-backbone cyclic peptides 4. Synthesis of protected N-alpha-functionalized alkylamino acids by reductive alkylation of natural amino acids. J. Chem. Soc., Perkin Trans. I, 1501-1510.
3. Cabilly, S., J. Heldman, E. Heldman, and E. Katchalski-Katzir. 1998a. The use of combinatorial libraries to identify ligands that interact with surface receptors in living cells. Methods Mol Biol. 87:175-183.
4. Cabilly, S., J. Heldman, and E. Katchalski-Katzir. 1998b. Screening phage display peptide libraries on nitrocellulose membranes. Methods Mol Biol. 87:185-194.
5. Cesareni, G., L. Castagnoli, and G. Cestra. 1999. Phage displayed peptide libraries. Comb Chem High Throughput Screen. 2:1-17.
6. Cortese, R., F. Felici, G. Galfre, A. Luzzago, P. Monaci, and A. Nicosia. 1994. Epitope discovery using peptide libraries displayed on phage. Trends Biotechnol. 12:262-267.
7. De Boever, J., Vandercjhove, D. and Kohen, F. 1989. Development of a chemiluminescence immunoassay for salivary progesterone using microtiter plates as a solid phase. Analytic Chimica Acta. 227: 119-127.
8. Devlin, J. J., P., L. C. and Devlin P. E. 1990. Random peptide libraries: a source of specific binding molecules. Science. 249:404-406.
9. DeWitt, N. 1999. Mapping protein interactions by phage display [In Process Citation]. Nat Biotechnol. 17:1150.
10. Giebel, L. B., Cass R. T., Milligan, D. L., Young, D. C., Arze, R. and Johnson, C. R. 1995. Screening of cyclic peptide phage libraries identifies ligands that bind streptavidin with high affinities. Biochemistry. 34:15430-15435.
11. Gilon, C., Halle, D., Chorev, M., Selinger, Z. and Byk, G. 1991. Backbone cyclization: a new method for conferring conformational constraint on peptides. Biopolymers 31, 745-750.
12. Green P S and Simpkins, J W. 2000. Estrogen and estrogen-like non-feminizing compounds. Their role in the prevention and treatment of Alzheimer's disease. Ann N Y Acad Sci. 924: 93-98.
13. Greene, G. L., and M. F. Press. 1986. Structure and dynamics of the estrogen receptor. J Steroid Biochem. 24:1-7.
14. Kohen, F. a. L., S. 1986. Monoclonal Antibodies to Steroid Hormones. In: Monoclonal Antibodies: Basic Principles, Experimental and Clinical Applications in Endocrinology, ed .G. Forti, M. B. Lipsett and M. Serio. Raven Press, pp. 87-95.
15. Kuiper, G. G., B. Carlsson, K. Grandien, E. Enmark, J. Haggblad, S. Nilsson, and J. A. Gustafsson. 1997. Comparison of the ligand binding specificity and transcript tissue distribution of estrogen receptors alpha and beta. Endocrinology. 138:863-870.
16. Kuiper, G. G., E. Enmark, M. Pelto-Huikko, S. Nilsson, and J. A. Gustafsson. 1996. Cloning of a novel receptor expressed in rat prostate and ovary. Proc Natl Acad Sci USA. 93:5925-5930.
17. Kuiper, G. G., and J. A. Gustafsson. 1997. The novel estrogen receptor-beta subtype: potential role in the cell- and promoter-specific actions of estrogens and anti-estrogens. FEBS Lett. 410:87-90.
18. Lowman, H. B. 1997. Bacteriophage display and discovery of peptide leads for drug development. Annu Rev Biophys Biomol Struct. 26:401-424.
19. Malnick, S. D., Shaer, A., Soreq, H. and Kaye, A. M. 1983. Estrogen induced creatine kinase in the reproductive system of the immature female rat. Endocrinology. 113:1907-1909.
20. Moe, G. R., S. Tan, and D. M. Granoff. 1999. Molecular mimetics of polysaccharide epitopes as vaccine candidates for prevention of Neisseria meningitidis serogroup B disease. FEMS Immunol Med Microbiol. 26:209-226.
21. Munoz D G and Feldman H. 2000. Causes of Alzheimer's disease. CMAJ. 162 (1):65-72.
22. Nillson, S., Kuiper, G., and Gustafsson, J A. 1998. ER beta a Novel Estrogen Receptor Offers the Potential for New Drug Development. Trends in Endocrinology and Metabolism. 9:387-395.
23. Qiu, J., P. Luo, K. Wasmund, Z. Steplewski, and T. Kieber-Emmons. 1999. Towards the development of peptide mimotopes of carbohydrate antigens as cancer vaccines. Hybridoma. 18:103-112.
24. Saggio, I. a. L., R. 1993. Biotin binders selected from a random peptide library expressed on phage. Biochem. J. 293:613-616.
25. Sapolsky R M and Finch C E. 2000. Alzheimer's disease and some speculations about the evolution of its modifiers. Ann N Y Acad Sci. 924: 99-103.
26. Scott, J. K., and G. P. Smith. 1990. Searching for peptide ligands with an epitope library. Science. 249:386-390.
27. Smith, G. P., and J. K. Scott. 1993. Libraries of peptides and proteins displayed on filamentous phage. Methods Enzymol. 217:228-257.
28. Somjen, D., Amir-Zaltsman, Y., Mor, G., Gayer, B., Lichter, S., Nevo, N. anf Kohen, F. 1998. A Monoclonal Antibody to Oestradiol Potentiates the Stimulation of the Specific Activity of the Brain Type Creatine Kinase by Oestrogen in vivo and in vitro. J Steroid Biochem. Mol. Biol. 64:297-304.
29. Somjen, D., Z. Y. Amir, G. Mor, B. Gayer, S. Lichter, G. Barnard, and F. Kohen. 1996. Anti-idiotypic antibody as an oestrogen mimetic in vivo: stimulation of creatine kinase specific activity in rat animal models. J Endocrinol. 149:305-312.
30. Strassburger, C. J. and Kohen, F. Two-site and Competitive Chemiluminescent Immunoassays. 1990. Methods Enzymol. 178: 481-496.
31. Venkatesh, N., S. H. Im, M. Balass, S. Fuchs, and E. Katchalski-Katzir. 2000. Prevention of passively transferred experimental autoimmune myasthenia gravis by a phage library-derived cyclic peptide [In Process Citation]. Proc. Nation. Acad. Sci. 97:761-766.
32. Walter, P., S. Green, G. Greene, A. Krust, J. M. Borneit, J. M. Jeltsch, A. Staub, E. Jensen, G. Scrace, M. Waterfield, and et al. 1985. Cloning of the human estrogen receptor cDNA. Proc Natl Acad Sci USA. 82:7889-7893.
33. Wang L., Andersson S., Warner M., Gustafsson, J A. 2001. Morphological abnormalities in the brains of estrogen receptor beta knockout mice. Proc Natl Acad Sci USA. 98(5): 2792-96.
34. Warner, M., S. Nilsson, and J. A. Gustafsson. 1999. The estrogen receptor family. Curr Opin Obstet Gynecol. 11:249-254.
35. Wise, P M. 2000. Estradiol: a protective factor. 13 Suppl 6: 1425-29.
36. Yayon, A., D. Aviezer, M. Safran, J. L. Gross, Y. Heldman, S. Cabilly, D. Givol, and E. Katchalski-Katzir. 1993. Isolation of peptides that inhibit binding of basic fibroblast growth factor to its receptor from a random phage-epitope library. Proc Natl Acad Sci USA. 90:10643-10647.

1. (Cancelled)

2. A synthetic peptide that mimics the biological activity of a steroid hormone, said synthetic peptide being selected from the group consisting of:
(i) a peptide exhibiting a steroid hormone-like biological activity;
(ii) a peptide obtained from (i) by deletion of one or more amino acid residues;
(iii) a peptide obtained by addition to a peptide (i) or (ii) of one or more natural or non-natural amino acid residues;
(iv) a peptide obtained by replacement of one or more amino acid residues of a peptide (i) to (iii) by the corresponding D-stereomer, by another natural amino acid residue or by a non-natural amino acid residue;
(v) a chemical dervative of a peptide (i) to (iv);
(vi) a cyclic derivative of a peptide (i) to (v);
(vii) a dual peptide consisting of two of the same or different peptides (i) to (vi), wherein the peptides are covalently linked to one another directly or through a spacer; and
(viii) a multimer comprising a number of the same or different peptides (i) to(vi).

3. The peptide according to claim 2, wherein said peptide has estrogenic-like activity and mimics the activity of an estrogen selected from the group consisting of estradiol, estrone or estriol.

4. (Cancelled)

5. The peptide according to claim 3, wherein said estrogen is estradiol and said peptide having estrogenic-like activity has at least 6 amino acid residues of the sequence:
X₄-X₂-Trp-Phe-X₁-Glu-X₃
wherein
X₁ is Phe or Tyr;
X₂ is Lys-Arg-, Ala-Arg, Lys-Ala-, Val-Arg-, Lys-Pro-, Val-Ser-, or Ile-Arg-;
X₄ is hydrogen or Thr-, Pro-Thr-, Asp-Pro-Thr-, Leu-Asp-Pro-Thr-, Ala-Leu-Asp-Pro-Thr-, Pro-Ala-Leu-Asp-Pro-Thr-, Leu-Pro-Ala-Leu Asp-Pro-Thr-, or Cys-Ala-Glu-Leu-Pro-Ala-Leu-Asp-Pro-Thr; and
X₃ is hydroxyl, Thr, -Thr-Lys, or -Thr-Lys-Pro-Pro-Pro-Pro-Cys;
and cyclic derivatives thereof.

6. The peptide according to claim 5, selected from the group consisting of the peptides:

| | |
|---|---|
| Leu-Pro-Ala-Leu-Asp-Pro-Thr-Lys-Arg-Trp-Phe- | (1) |
| Phe-Glu-Thr-Lys | |
| Cyclic [Cys-Ala-Glu-Leu-Pro-Ala-Leu-Asp-Pro- | (2) |
| Thr-Lys-Arg-Trp-Phe-Phe-Glu-Thr-Lys-Pro-Pro- | |
| Pro-Pro-Cys] | |
| Lys-Arg-Trp-Phe-Phe-Glu | (44) |
| Ala-Arg-Trp-Phe-Phe-Glu | (A43) |
| Lys-Ala-Trp-Phe-Phe-Glu | (A44) |
| Val-Arg-Trp-Phe-Phe-Glu | (39) |
| Lys-Pro-Trp-Phe-Phe-Glu | (21) |
| Val-Ser-Trp-Phe-Phe-Glu | (B34) |
| Ile-Arg-Trp-Phe-Phe-Glu | (B37) |

7. A peptide according to claim 2, wherein said peptide has progestational-like activity and mimics the activity of progesterone.

8. (Cancelled)

9. The peptide according to claim 7, wherein said peptide having progesterone-like activity is selected from the group consisting of the peptides 3 and 4, of the sequences:

| | |
|---|---|
| Val-Asn-His-Pro-Trp-Asp-Gln-Ala-Gln-Phe-Leu- | (3) |
| Ser-Thr-Ile | |
| Ser-Asn-Pro-Phe-Cys-Gln-Thr-Asp-Gly-Asp-Cys- | (4) |
| His-Val-His-Thr | |

10. A peptide according to claim 2, wherein said peptide has androgenic-like activity and mimics the activity of testosterone.

11. (Cancelled)

12. A peptide according to claim 2, wherein said peptide has adrenocorticoid-like activity and mimics the activity of an adrenocorticoid hormone selected from the group consisting of cortisone, hydrocortisone or corticosterone.

13. (Cancelled)

14. A pharmaceutical composition comprising a peptide according to claim 2, and a pharmaceutically acceptable carrier.

15. A pharmaceutical composition comprising a peptide according claim 3, and a pharmaceutically acceptable carrier for the purpose of estrogen replacement therapy.

16. A pharmaceutical composition according to claim 15, for treatment of hormone-dependent cancers selected from the group consisting of breast, prostate and colon cancer, postmenopausal symptoms or prevention and/or treatment of osteoporosis.

17. A pharmaceutical composition according to claim 15, for prevention and/or treatment of degenerative diseases of central nervous system.

18. A pharmaceutical composition according to claim 17, for prevention and/or treatment of Alzheimer's disease, Parkinson's disease or another degenerative disease of the central nervous system resulting from trauma or stroke in the brain.

19. A pharmaceutical composition comprising a peptide according to claim 7, and a pharmaceutically acceptable carrier for the purpose of contraception or in endocrine therapy of breast cancer, uterine fibroids or polycystic ovary syndrome.

20. (Cancelled)

21. (Cancelled)

22. (Cancelled)

23. (Cancelled)

24. (Cancelled)

25. (Cancelled)

26. (Cancelled)

27. (Cancelled)

28. (Cancelled)

29. A method for screening a combinatorial peptide library for the identification of a peptide exhibiting a steroid hormone biologic-like activity, which comprises:
(i) providing a monoclonal antibody with high affinity and specificity to the steroid hormone investigated;
(ii) screening a combinatorial peptide library with said monoclonal antibody of (i) for the identification of peptides that bind specifically to said monoclonal antibody;
(iii) isolating said peptides and testing them for competition with said steroid hormone for binding to said monoclonal antibody in vitro and for binding to said steroid hormone receptor in vitro; and
(iv) identifying the peptides that mimic said steroid hormone activity as being those that successfully compete with said steroid hormone for binding to its monoclonal antibody and receptor.

30. A conjugate of a peptide according to claim 2 with a label selected from the group consisting of a fluorescent, a paramagnetic and a radioactive marker.

31. A conjugate according to claim 30, wherein said peptide is a peptide that has estrogen-like activity and that mimics the activity of an estrogen selected from the group consisting of estradiol, estrone or estriol.

32. A conjugate of a peptide according to claim 2 with a chemotherapeutic drug.

33. A conjugate of a peptide according to claim 3 with a chemotherapeutic drug.


SEQUENCE LISTING (36 sequences)

SEQ ID NO: 1 - protein, 15 aa, Artificial
  Synthetic
LPALDPTKRWFFETK

SEQ ID NO: 2 - protein, 23 aa, Artificial
  Synthetic
CAELPALDPTKRWFFETKPPPPC

SEQ ID NO: 3 - protein, 7 aa, Artificial
  Synthetic
XXWFXEX

SEQ ID NO: 4 - protein, 6 aa, Artificial
  Synthetic
KRWFFE

SEQ ID NO: 5 - protein, 6 aa, Artificial
  Synthetic
ARWFFE

SEQ ID NO: 6 - protein, 6 aa, Artificial
  Synthetic
KAWFFE

SEQ ID NO: 7 - protein, 6 aa, Artificial
  Synthetic
VRWFFE

SEQ ID NO: 8 - protein, 6 aa, Artificial
  Synthetic
KPWFFE

SEQ ID NO: 9 - protein, 6 aa, Artificial
  Synthetic
VSWFFE

SEQ ID NO: 10 - protein, 6 aa, Artificial
  Synthetic
IRWFFE

SEQ ID NO: 11 - protein, 6 aa, Artificial
  Synthetic
IRWFFE

SEQ ID NO: 12 - protein, 14 aa, Artificial
  Synthetic
VNHPWDQAQFLSTI

SEQ ID NO: 13 - protein, 15 aa, Artificial
  Synthetic
SNPFCQTDGDCHVHT

SEQ ID NO: 14 - protein, 15 aa, Artificial
  Synthetic
AHWNSENTVVGLPSK

SEQ ID NO: 15 - protein, 15 aa, Artificial
  Synthetic
GMQMHQRHVYLSKRP

SEQ ID NO: 16 - protein, 14 aa, Artificial
  Synthetic
LPALDPTKRWFFET

SEQ ID NO: 17 - protein, 13 aa, Artificial
  Synthetic
LPALDPTKRWFFE

SEQ ID NO: 18 - protein, 12 aa, Artificial
  Synthetic
LPALDPTKRWFF

SEQ ID NO: 19 - protein, 11 aa, Artificial
  Synthetic
LPALDPTKRWF

SEQ ID NO: 20 - protein, 14 aa, Artificial
  Synthetic
PALDPTKRWFFETK

SEQ ID NO: 21 - protein, 13 aa, Artificial
  Synthetic
ALDPTKRWFFETK

SEQ ID NO: 22 - protein, 12 aa, Artificial
  Synthetic
LDPTKRWFFETK

SEQ ID NO: 23 - protein, 11 aa, Artificial
  Synthetic
DPTKRWFFETK

SEQ ID NO: 24 - protein, 10 aa, Artificial
  Synthetic
PTKRWFFETK

SEQ ID NO: 25 - protein, 9 aa, Artificial
  Synthetic
TKRWFFETK

SEQ ID NO: 26 - protein, 8 aa, Artificial
  Synthetic
PTKRWFFE

SEQ ID NO: 27 - protein, 7 aa, Artificial
  Synthetic
TKRWFFE

SEQ ID NO: 28 - protein, 5 aa, Artificial
  Synthetic
RWFFE

SEQ ID NO: 29 - protein, 4 aa, Artificial
  Synthetic
WFFE

SEQ ID NO: 30 - protein, 6 aa, Artificial
  Synthetic
KRAFFE

SEQ ID NO: 31 - protein, 6 aa, Artificial
  Synthetic
KRWAFE

SEQ ID NO: 32 - protein, 6 aa, Artificial
  Synthetic
KRWFAE

SEQ ID NO: 33 - protein, 6 aa, Artificial
  Synthetic
KSWFFE

SEQ ID NO: 34 - protein, 6 aa, Artificial
  Synthetic
VPWFFE

SEQ ID NO: 35 - protein, 6 aa, Artificial
  Synthetic
VRWFYE

SEQ ID NO: 36 - protein, 6 aa, Artificial
  Synthetic
LRWFFE